(19) United States
(12) Patent Application Publication
(10) Pub. No.: US 2018/0055427 A1
(43) Pub. Date: Mar. 1, 2018

(54) Method and Apparatus to Enhance Peripheral Venous Oxygen Measurements

(71) Applicant: TIRO Medical Limited, Christchurch (NZ)

(72) Inventors: J. Geoffrey Chase, Christchurch (NZ); Christopher G. Pretty, Christchurch (NZ); Geoffrey M. Shaw, Christchurch (NZ); Lawrence A. Ray, Rochester, NY (US); Richard William Wien, Pittsford, NY (US)

(73) Assignee: TIRO Medical Limited, Christchurch (NZ)

(21) Appl. No.: 15/674,739

(22) Filed: Aug. 11, 2017

Related U.S. Application Data
(60) Provisional application No. 62/378,743, filed on Aug. 24, 2016.

(51) Int. Cl. A61B 5/1455; A61B 5/00; A61B 5/1491; A61B 5/1495

(52) U.S. Cl. CPC A61B 5/14551; A61B 5/6826; A61B 5/1495; A61B 5/1491; A61B 5/0053

(57) ABSTRACT

A method and apparatus for non-invasive measure of venous oxygen saturation, which can be useful for early diagnosis of microcirculatory dysfunction and treatment of medical conditions, such as septic shock, is disclosed. The method applies external stimulus to a patient to improve the signals to an oximeter yielding more reliable measurements. The device implementing the method uses superficial low frequency and low pressure pulse to the patient near the site of the oximeter as well as optionally controlling the patient's temperature near the site of the oximeter. The results also include non-invasive measurements of SpaO₂, SpvO₂ and O_{2E}.

### CROSS REFERENCE

This application claims the benefit of the filing date of U.S. Provisional Patent Application Ser. No. 62/378743, filed Aug. 24, 2016, which is hereby incorporated by reference in its entirety.

### FIELD

This disclosure relates to a method and apparatus to enhance peripheral venous oxygen measurements, and in particular, to enhance peripheral venous oxygen measurements by controlling the application of pressure, frequency and optionally temperature.

### BACKGROUND

Pulse oximeters are the ubiquitous devices used in hospitals to non-invasively estimate arterial blood oxygen saturation (SpO₂) and monitor heart rate (HR). Pulse oximetry uses photoplethysmograph (PPG) signals acquired by a sensor, typically mounted on a finger, toe, or ear-lobe to optically detect blood volume changes in the tissue. The PPG results from the time-varying amount of blood that is pushed into the vascular bed during systole and diastole. Conventional pulse oximetry relies on the pulsatile nature of arterial blood and differential absorption of oxyhaemoglobin and de-oxyhaemoglobin at red (RD) and infrared (IR) wavelengths to estimate SpO₂ and HR.

Commercial pulse oximeter probes consist of two high output RD and IR light emitting diodes (LED) and a very sensitive photo-detector (PD). Light energy transmitted through tissue is detected by the PD, which generates the PPG signal. From the PPG signal, the slowly changing (DC) and the rapidly changing (AC) signals are extracted. The DC signal predominantly captures the unchanging light scattering and absorption. The AC signal predominantly captures the varying absorption due to pulsatile arterial blood and is synchronous with HR. Thus, by taking the appropriate AC/DC ratios and calibration, SpO₂ can be reliably estimated.

While the predominant application of pulse oximeters has been arterial blood oxygen saturation (SaO₂) estimation, the raw PPG waveform (PPG_{Raw}) is rich with relevant physiological information. Indeed, the PPG_{Raw} contains a complex mixture of the influences of arterial, venous, autonomic and respiratory systems on the peripheral circulation. Non-invasive estimation of blood flow change in muscles and bones using PPG was previously reported showing that the AC component of the PPG corresponds to blood flow while the DC corresponds to the blood volume change. Thus, application of PPG is not restricted to SpO₂ estimation.

A number of factors have been reported to limit pulse oximeter accuracy, including motion artifacts, environmental noise, skin tone, gender, nail polishes, and ambient light. Another important limiting factor is temperature, which is often overlooked. It is generally accepted that cold digits may provide inaccurate pulse oximeter readings. People with naturally very cold fingers or patients in intensive care units, where room temperature is maintained at 20° C., are examples of cases that can be affected by this problem.

Commercially available pulse oximeters have limited access to raw PPG signal data and thus the rate of new pulse oximeter applications has diminished significantly. Although conventional pulse oximeters use two wavelengths, only the infrared PPG waveforms are typically presented to the user or clinician. In addition, the filtering of a majority of the DC signal removes valuable physiological data, including venous blood absorption information. The current systems do not allow for temperature control of the site on the body being measured.

The pulse oximeter is one of the most widely accepted non-invasive clinical monitoring systems. However, conventional pulse oximeters are restricted to estimation of SaO₂ only. Coupling estimation of peripheral venous blood oxygen saturation (SvO₂) with estimation of SaO₂ would enable assessment of local oxygen extraction (O₂E) according to (O₂E=SaO₂−SvO₂) and any related metrics. O₂E information could provide an indicator of the adequacy of local tissue perfusion, to aid in early diagnosis of microcirculatory dysfunction in medical conditions, such as sepsis and shock.

Venous blood in the periphery is typically non-pulsatile in nature and thus conventional pulse oximetry cannot be used to measure SvO₂. Arteries and veins have significantly different vessel wall mechanical properties. Venous walls are significantly thinner and less elastic than arterial walls. Due to the significant differences in mechanical properties of the two vessels, under low pressure, the veins are up to 10-20 times more compliant compared to arteries. Vessel compliance (C) is the ability of a blood vessel to distend and increase in volume with increasing transmural pressure, as defined:

$$C = \frac{Δ V}{Δ P}$$

Transmural pressure (ΔP) is the pressure difference between the inside and outside of the vessel wall. With small changes in pressure, the circulating blood inside the veins thus experiences large volume changes compared to the arteries. This large compliance difference is what causes the respiratory modulations in the venous blood seen in previous studies.

U.S. Pat. No. 7,263,395 discloses an automated artificial pulse generation (APG) system which generates user-defined pulsations that can mechanically modulate blood volumes inside the finger. This system is designed to utilize the arterial-venous compliance difference to induce artificial pulsations predominantly in the venous compartment. A pressure transducer is applied to a first site on a body and an oximeter is applied to a second site. Ideally, the magnitude of the pulsations is controlled such that the venous blood system is modulated without disturbing the arterial system. However, the current methods use an activation signal that has a frequency (above 4 Hz) and pressure (160 mm Hg) close to normal arterial signals. These two signals can easily be confounded and a significant effort is needed to disentangle the two signals. Moreover, the same correlation formula is used to calculate both the venous and arterial oxygen saturation. No mention of regulation of measurement temperature is disclosed or suggested. There is a need to have an activation signal that is sufficiently distinguished from the arterial signal to enable simpler and more robust signal processing.

Thus, there is a need for a pulse oximeter system that overcomes the limitations of current pulse oximeters, including controlling the temperature of the patient's fingers. Moreover, providing a method to generate an artificial pulse which avoids confounded signals and significant effort to disentangle the two signals would enable additional diagnostic and real-time measurement of patient data.

### SUMMARY

In accordance with an aspect of the present invention, there is provided a method for non-invasively measuring venous oxygen saturation of a subject, including: applying a pressure transducer at a first site on a subject having venous and arterial systems; applying a drive signal to the pressure transducer at a frequency below about 0.83 Hz to cause a series of pulsations of a magnitude in the venous blood volume less than diastolic pressure of the subject such that the arterial system is minimally disturbed; applying an oximeter device at a second site on the subject and optionally controlling the temperature; measuring output signals received from the oximeter device, the output signals containing a component representative of the modulation of venous blood volume due to the series of pulsations; and deriving a measure of venous oxygen saturation of the subject from the frequency response of the output signals.

In accordance with another aspect of the present disclosure, there is provided an apparatus for non-invasively measuring venous oxygen saturation of a subject, including: a pressure transducer capable of applying a series of pulsations to a first site on a subject having venous and arterial systems; a pulse oximeter; optionally a heat source; optionally a temperature sensor or a thermometer; a controller in communication with the pressure transducer, pulse oximeter, optional heat source and optional temperature sensor or thermometer, wherein the controller is capable of applying a drive signal to the pressure transducer at a frequency below about 0.83 Hz to cause a series of pulsations of a magnitude in the venous blood volume less than diastolic pressure such that the arterial system is minimally disturbed, optionally controlling the temperature of the subject at a second site, and measuring output signals from the pulse oximeter, the output signals containing a component representative of the modulation of venous blood volume due to the series of pulsations; and a signal processor capable of extracting a value for venous oxygen saturation from the frequency response of the output signals.

These and other aspects of the present disclosure will become apparent upon a review of the following detailed description and the claims appended thereto.

### BRIEF DESCRIPTION OF THE DRAWINGS

FIG. 1 is a schematic diagram of a system in accordance with an embodiment of the present disclosure;

FIG. 2 is a block diagram of an APG system in accordance with an embodiment of the present disclosure;

FIG. 3 is a flowchart of a method in accordance with an embodiment of the present disclosure;

FIG. 4A (AC signal at RD), FIG. 4B (AC signal at IR), FIG. 4C (APG signal at RD), FIG. 4D (APG signal at IR), FIG. 4E (DC signal at RD), and FIG. 4F (DC signal at IR) are frequency power spectrums in accordance with the Example of the present disclosure; and

FIG. 5. is a graph illustrating the correlation between estimated R_{Ven} and measured SvO₂ and the correlation between R_{Art} and SpvO₂ generated by the Example data.

### DETAILED DESCRIPTION

This disclosure provides a method and apparatus to improve the real-time patient data collection by adding to and improving the signal quality of the data collected by a pulse oximeter. The disclosure enables one to measure patient pulse-oximetry as well as optionally a way to control the patient temperature near the point where the pulse oximeter is attached and a way to artificially generate a low frequency pulse to ascertain the arterial-venous compliance difference in an efficient manner.

Recent work (see Kahn, et. al., “Investigating the Effects of Temperature on Photoplethysmography,” IFAC-PapersOnLine, 2015-Elsevier) has shown large increases in PPG quality and pulse oximeter accuracy when the temperature of fingers is controlled to near body temperature (30° C. to 35° C.).

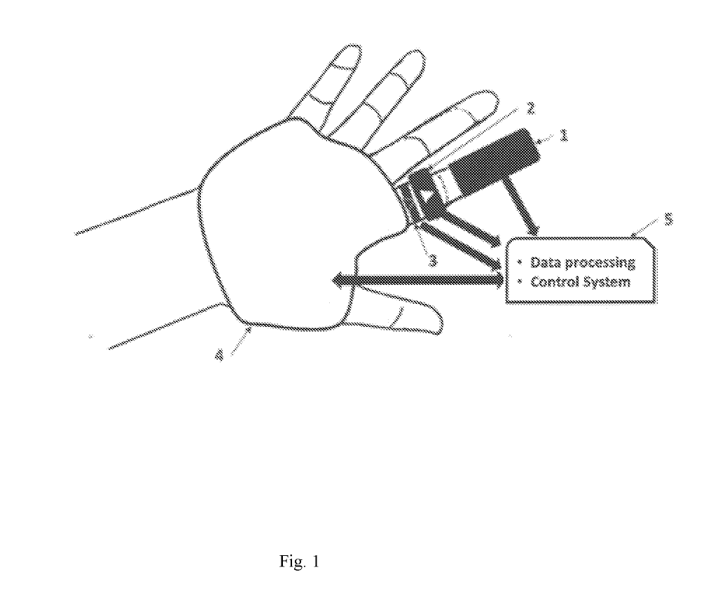

In an embodiment shown in FIG. 1, a pulse oximeter can be used for peripheral SvO₂ estimation, employing low frequency (<1 Hz) modulations to the peripheral vasculature to utilize the difference in the mechanical properties of arteries and veins. These modulations are then detected by PPG and analysed to assess regional SvO₂. Successful estimation of SvO₂ combined with SaO₂ enables pulse oximeter based estimation of O₂E.

An embodiment of a device, shown in FIG. 1, includes a fingerless thermal glove 4 and a pressure controlled digit cuff 2 that is placed around a finger (analogous to a ring) that can be pulsed at a low frequency. The system includes an optoelectronic sensor 1 which contains light sources (typically LED with output from 600 to 1000 nanometers) and a photo-detector. A control system 5 provides control of output from the LED as well as time demultiplexing for the light sources. Output from detector in the optoelectronic sensor 1 goes to the control system 5 for data processing. An artificial pressure generation (APG) system includes the digit cuff whose inflation pressure and frequency of inflation are controlled by the control system 5. Optionally, a temperature sensor 3 is connected to the control system 5 to provide temperature measurements. The thermal glove 4 in an embodiment provides heating to maintain a skin temperature in a suitable range of from about 30° C. to about 35° C. and is controlled by control system 5 based on feedback from the thermal glove. Control system 5 provides data processing which incorporates the correlation equation in accordance with the present invention which exhibits a linear relationship between the R value and venous oxygen saturation providing the ability to derive estimates of venous oxygen saturation, arterial oxygen saturation and oxygen extraction.

Optoelectronic Sensor: A suitable optoelectronic sensor includes a standard transmission mode sensor used to acquire PPG data from a finger. The dual LED sensor produces 660 nm and 940 nm wavelength light for the RD and IR modes, respectively, and contains a high sensitivity PD.

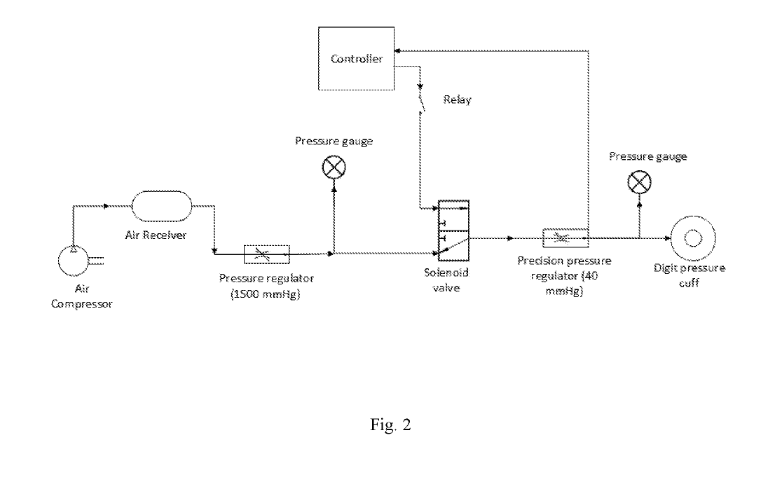

Artificial Pulse Generator: In accordance with an embodiment, an APG system is shown schematically in FIG. 2. By periodically inflating and deflating the digit cuff, an artificial respiration-like pulse can be modulated onto the venous blood of the finger. A suitable modulation pressure includes a pressure below the patient diastolic pressure; a pressure below about 80 mm HG; a pressure of from about 10 mm Hg to about 80 mm Hg; or a pressure of about 40 mm Hg to avoid interference with arterial flow and the compression pressure can be felt exclusively in the venous compartment.

The system is driven, for example, utilizing an air source, such as, compressed air that is stored and if needed restored by an air compression unit. The regulator assures the air pressure transmitted to the pressure cuff is adequate to inflate the cuff, while not allowing so much air pressure to either cause the patient needless discomfort or local tissue trauma. Since the intent of the cuff is to send a pulse this can be accomplished by alternately allowing the cuff to inflate and to deflate. A suitable modulation frequency includes a frequency below about 0.83 Hz, time for this cycle to complete is approximately 50 times a minute; a frequency of from about 0.1 Hz to about 0.83 Hz; a frequency of from about 0.2 Hz to about 0.4 Hz; or a frequency of about 0.2 Hz. The control of this cycle can be done by a small electronic controller. This controller may be part of the overall system controller.

As a result of the APG activation, the original PPG waveform contains an additional pulsatile signal, superimposed on the DC signal. The effect of introducing a pulsatile signal can be considered as an artefact introduced into the PPG signal. This low frequency artefact was spectrally extracted and analyzed for SvO₂.

Thermal Glove: The temperature sensor and thermal glove provides the ability to maintain the patient's body temperature near the optoelectronic sensor at a temperature of above about 30° C. or preferably within a temperature range of from about 30° C. to about 35° C. This can be accomplished, for example, as shown in FIG. 1, by a temperature sensor 3 in contact with the patient's hand and a thermal glove 4 containing a heating capability. The temperature sensor 3 is connected to a control system 5 which monitors the signal and modifies the current sent to the thermal glove to either increase, maintain or reduce (including setting to zero) the heat produced by the glove. Thermal gloves are well known and are used for many reasons though the control of the temperature is usually performed by the wearer of the glove, as in most cases the wearer is conscience and is performing some other task. The glove exposes the fingers of the patient to permit the APG to be attached as well as the optoelectronic sensor. Like the APG, the control of the thermal glove can be managed by the overall system controller.

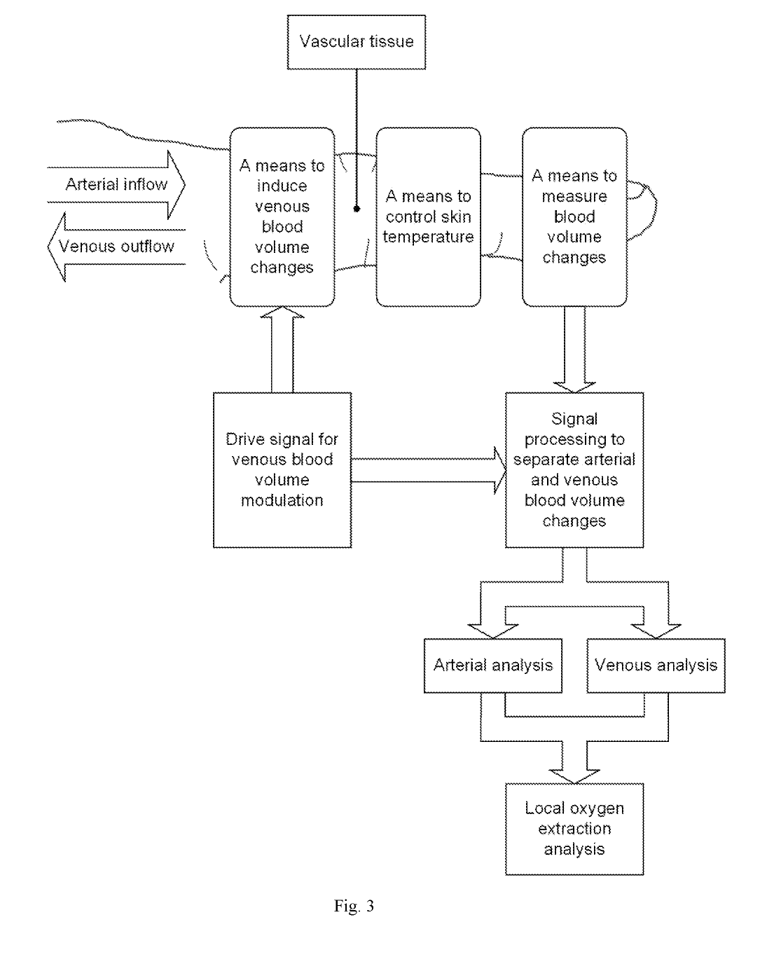

Data Processing and Control System: This system, an embodiment as shown in accordance with the method of FIG. 3 performs two major functions. The first is the control of the devices attached to the system, such as the APG. The other is the analysis of the data collected and transmitted by the optoelectronic sensor. The first functions have been described previously. The second function utilizes Walton's time domain equations (see Walton, et. al., Measuring venous oxygenation using the photoplethysmograph waveform. J Clin Monit Comput, 24, 4, 295-303.), which are used to calculate the ratio of ratios (R values) relating to arterial (R_{Art}) and venous (R_{Ven}) oxygen saturations, as modified below:

$${R}_{Art} = \frac{{( {\vert A C \vert}_{HR} / {\vert D C \vert}_{0 H z} )}_{RD}}{{( {\vert A C \vert}_{HR} / {\vert D C \vert}_{O H z} )}_{IR}}$$

$${R}_{Ven} = \frac{{( {\vert APG \vert}_{H z} / {\vert D C \vert}_{0 H z} )}_{RD}}{{( {\vert APG \vert}_{H z} / {\vert D C \vert}_{O H z} )}_{IR}}$$

Where,
   |AC|_{HR} is the peak magnitude at HR frequency of the AC signal;
   |APG|_{Hz} is the peak magnitude at the applied pulse frequency of the APG signal; and
   |DC|_{0 Hz} is the peak magnitude at 0 Hz frequency of the DC signal.

The values for arterial and venous saturation can then be found using the following calibration equations. It should be noted that the present system does not use identical coefficients for Rₐᵣₜ and Rᵥₑₙ as was previously done in prior systems, but rather the formulas below:

SpaO₂=110−25*R_{Art}

SpvO₂=110−40*R_{Ven}

wherein SpaO₂ is the pulse oximeter estimation of SaO₂ and SpvO₂ is the pulse oximeter estimation of SvO₂

The peak magnitude may be calculated using peak and trough detection algorithms or by using FFT based frequency domain analysis.

Determination of calibration equations: In order to ascertain the coefficients used in the calibration equations a study was conducted using a cohort of volunteers in order to collect sufficient data using a system in accordance with the present disclosure and traditional clinical measurement methods as set forth in the Example. An APG system was developed to exploit the significant arterial-venous compliance difference to induce artificial pulsations predominantly in the venous compartment. An embodiment of the APG system is shown schematically in FIG. 2. This system uses a pneumatic UDC2.5 (D. E. Hokanson Inc., Bellevue, Wash., USA) digit cuff placed at the intermediate phalanges of the middle finger, as shown in FIG. 1, to mechanically modulate venous blood in the finger. By periodically inflating and deflating the digit cuff, an artificial respiration-like pulse can be induced onto the venous blood of the finger.

### EXAMPLE

A study was conducted in the intensive care unit (ICU) of St George's Hospital, Christchurch. Eight healthy adult, male, volunteers (aged 23-37 years) with no pre-existing medical conditions were recruited, and provided signed, informed consent.

A catheter was inserted into a vein on the back of the left hand in order to get SvO₂ by standard methods. A portable i-Stat1 blood gas analyzer (Abbott, Princeton, N.J., USA) was employed to analyze catheter drawn blood samples. A standard transmission mode sensor 1 was connected to the custom pulse oximeter (PO) system and was clipped to the index/middle finger of the left hand, as illustrated in FIG. 1.

A temperature sensor 3 was attached next to the PO sensor 1 on the skin surface to continuously monitor digit skin temperature. Subjects were asked to refrain from smoking and strenuous physical activities for at least 4 hours prior to the experiment. During the study, subjects were comfortably seated, while resting their left arm on a flat surface at approximately the same height as their heart and with minimum movement. Tests were performed to achieve a wide range of experimentally measured SvO₂ values and provide a valid dataset in order to ascertain the calibration equation.

Inflation/deflation of the digit cuff 2 is controlled by a 3-way solenoid valve (EVT 307-5DZ-02F-Q, SMC, Noblesville, Ind., USA). Operation of the solenoid valve is managed by the controller 5. The solenoid is switched on and off with square pulses at 0.2 Hz and 50% duty cycle. The 0.2 Hz frequency is close to typical respiratory frequencies to create artificial venous blood modulations when detecting natural venous blood modulations in the oesophagus using a PPG sensor.

A precision pressure regulator (AS1002F, SMC, Noblesville, Ind., USA) was used to control the pressure of air supplied to inflate the cuff at 40-50 mmHg, which is approximately 50% below typical diastolic arterial pressures. Thus, this cuff compression pressure will predominantly effect the venous compartment. For the deflation portion of the pulse, the air in the cuff was exhausted to the atmosphere with the solenoid valve turning off.

PPG finger sensor control and data acquisition were managed with the PO system, which includes serial communication and a graphical interface. The PO system is based on the CY8CKIT-050 PSoC 5LP Development Kit (Cypress Semiconductor, San Jose, Calif., USA) that has an ARM Cortex M3 microcontroller. The PPG sensor uses 660 and 940 nm wavelengths for the RD and IR LEDs, respectively, and a very sensitive photodetector (PD). Feedback control was used by the PO system microcontroller to adjust LED intensities specific to finger thickness. Light energy received at the PD was converted to current signals and passed through a two stage trans-impedance amplifier for signal conditioning. A de-multiplexer splits the conditioned PPG signals so that the RD and IR PPGs are processed independently. Analog PPG signals in the range 0-5 V were sampled at 50 Hz by a 16-bit analog-to-digital converter (ADC) on the development board. Sampled data were sent to a PC via serial communication and recorded for offline signal processing in MATLAB (R2014a, MathWorks, Natick, Mass., USA).

A two-stage digital filter system was implemented to extract the PPG signals of interest from the raw and amplified PPG data. Zero phase (forward—reverse) filtering was applied at each filter stage to reduce phase distortion and maintain PPG waveform shape.

The first filter stage was an equiripple, low-pass, FIR filter with a cut-off frequency of 10 Hz and attenuation of 50 dB to effectively remove any unwanted, high frequency noise from the PPG signals. The second filter stage had three parallel filters to separately extract high frequency (AC), APG modulated frequency (APG), and very low frequency (DC) signals. An empirically derived 4th order band-pass, Butterworth, IIR filter extracted AC signals, such as the cardiac frequencies, with pass band frequencies of 0.67-4.5 Hz. An empirically derived Butterworth, IIR filter extracted APG signals with pass band frequencies of 0.15-0.67 Hz. An empirically derived Butterworth, IIR filter with a cut-off frequency of 0.15 Hz was used to extract DC signals between 0 and 0.15 Hz.

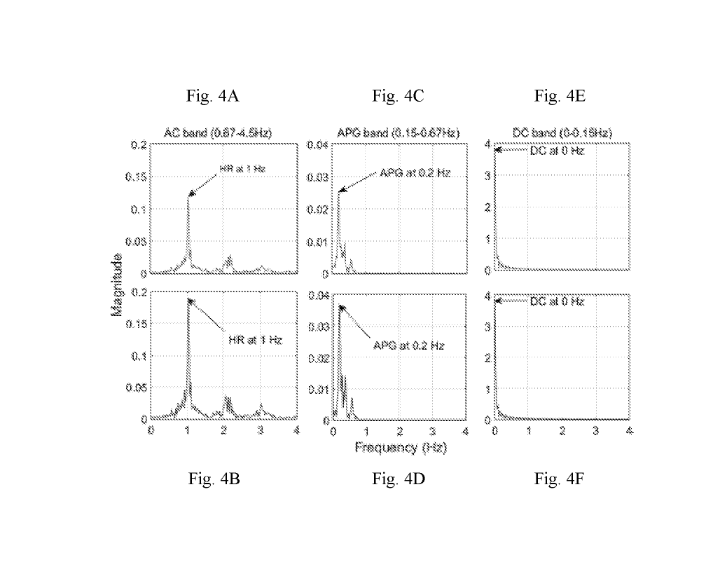

FIG. 4 shows the frequency domain magnitudes determined from the frequency power spectrum generated by one of the subjects as an example of the data generated in this experiment. The magnitude for the AC signals at HR frequency was higher than the APG signals at 0.2 Hz. This difference is due to the amplification of the AC signal during signal conditioning. In contrast, the APG signal has no prior amplification since it is extracted from the unamplified raw PPG data.

A Fast Fourier Transform (FFT) analysis was used to determine the relative power in each part of the PPG signal generated from the subjects to calculate the “modulation ratios” (R values):

$$\begin{matrix}{R}_{Art} = \frac{{( {\vert A C \vert}_{HR} / {\vert D C \vert}_{0 H z} )}_{RD}}{{( {\vert A C \vert}_{HR} / {\vert D C \vert}_{0 H z} )}_{IR}} & ( 1 ) \\ {R}_{Ven} = \frac{{( {\vert APG \vert}_{0.2 H z} / {\vert D C \vert}_{0 H z} )}_{RD}}{{( {\vert APG \vert}_{0.2 H z} / {\vert D C \vert}_{0 H z} )}_{IR}} & ( 2 )\end{matrix}$$

where,

R_{Art} is the modulation ratio related to arterial blood.

R_{Ven} is the modulation ratio related to venous blood.

|AC|_{HR} is the peak Fourier magnitude of the AC signal, related to cardiac frequency.

|APG|_{0.2 Hz} is the peak Fourier magnitude at 0.2 Hz of the APG signal.

|DC|_{0 Hz} is the peak Fourier magnitude at 0 Hz frequency of the DC signal.

The fundamental frequency associated with the peak magnitude was identified from the frequency power spectra of AC, APG, and DC offset signals. Equations 1 and 2 were then used to compute values of R_{Art} and R_{Ven}.

A linear model was fitted to the calculated R_{Art} and R_{Ven} values and measured blood gas SvO₂ to generate the correlation equation in accordance with the present invention:

SpaO₂=110−25*R_{Art}

SpvO₂=110−40*R_{Ven}

wherein SpaO₂ is the pulse oximeter estimation of SaO₂ and SpvO₂ is the pulse oximeter estimation of SvO₂

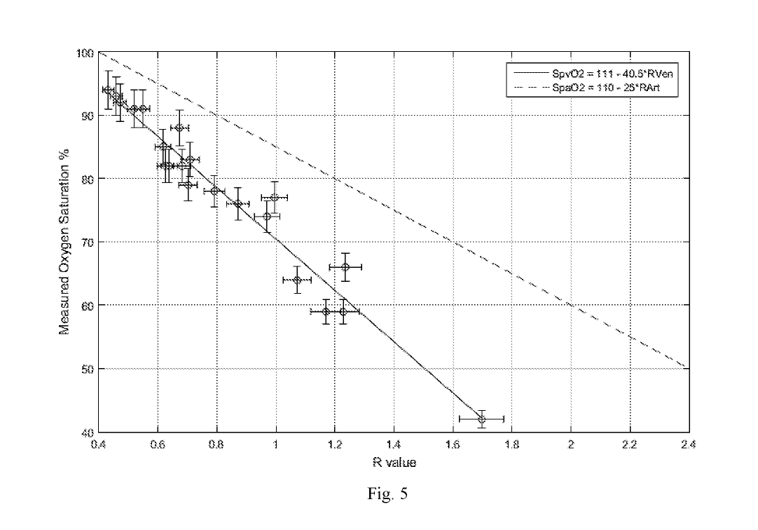

The coefficient of determination (r²) was computed to assess the strength of their correlation for both were found to have a sufficiently high degree of correlation. The linear models can now be used for the general population as the calibration curve to estimate SpvO₂ and SpaO₂. See FIG. 5, which summarizes the experimental data from the subjects and data processing that establishes the calibration equations from which venous oxygen saturation can be determined in accordance with the present invention.

This system presents a non-invasive method to induce venous blood modulations using a pneumatic digit cuff, which can be captured by PPG. A calibration model is derived from venous blood modulated PPG signals and measured SvO₂ to assess SpvO₂ estimate. In addition, the gradient of this model (−40%) is different to the gradient of the conventional pulse oximeter calibration model (−25%). The proposed SpvO₂ estimation model can be used as a potential metric and indicator for low O₂E, consumption, and tissue hypoxia.

Real-time estimation of peripheral SvO₂, using the pulse oximeter method in accordance with the present invention, and SaO₂ will enable continuous, real-time monitoring of these physiological conditions. Thus, improvement and application of this concept could aid in diagnosis of medical conditions related to microcirculation dysfunction, such as sepsis and cardiac failure, which are both common in the ICU, as well as length of stay, cost, and mortality.

Although various embodiments have been depicted and described in detail herein, it will be apparent to those skilled in the relevant art that various modifications, additions, substitutions and the like can be made without departing from the spirit of the disclosure and these are therefore considered to be within the scope of the disclosure as defined in the claims which follow.

What is claimed:

1. A method for non-invasively measuring venous oxygen saturation of a subject, comprising:
applying a pressure transducer at a first site on a subject having venous and arterial systems;
applying a drive signal to the pressure transducer at a frequency below about 0.83 Hz to cause a series of pulsations of a magnitude in the venous blood volume less than diastolic pressure of the subject such that the arterial system is minimally disturbed;
applying an oximeter device at a second site on the subject;
measuring output signals received from the oximeter device, the output signals containing a component representative of the modulation of venous blood volume due to the series of pulsations; and
deriving a measure of venous oxygen saturation of the subject from the frequency response of the output signals.

2. The method of claim 1, further comprising controlling the temperature of the subject at the second site.

3. The method of claim 2, wherein the temperature is above about 30° C.

4. The method of claim 2, wherein the temperature is within a range of from about 30° C. to about 35° C.

5. The method of claim 1, wherein the frequency is within a range of from about 0.1 Hz to about 0.83 Hz.

6. The method of claim 1, wherein the frequency is about 0.2 Hz.

7. The method of claim 1, wherein the magnitude of the series of pulsations is a pressure within a range of from about 10 mmHg to about 80 mmHg.

8. The method of claim 1, wherein the magnitude of the series of pulsations is a pressure of about 40 mmHg.

9. The method of claim 1, wherein the measure of venous oxygen saturation is derived from the following equations:
$${R}_{Ven} = \frac{{( {\vert APG \vert}_{H z} / {\vert D C \vert}_{0 H z} )}_{RD}}{{( {\vert APG \vert}_{H z} / {\vert D C \vert}_{0 H z} )}_{IR}} ,$$
wherein
R_{Ven} is the modulation ratio related to venous blood,

|APG|_{Hz} is the peak Fourier magnitude at the applied pulse frequency of the APG signal,

|DC|_{0 Hz} is the peak Fourier magnitude at 0 Hz frequency of the DC signal; and
SpvO₂=110−40*R_{Ven}
wherein SpvO₂ is the pulse oximeter estimation of SvO₂.

10. The method of claim 1, further comprising deactivating the pressure transducer and determining the arterial oxygen saturation.

11. The method of claim 10, wherein the determination of arterial and venous oxygen saturation use different calibration equations.

12. An apparatus for non-invasively measuring venous oxygen saturation of a subject, comprising:
a pressure transducer capable of applying a series of pulsations to a first site on a subject having venous and arterial systems;
a pulse oximeter;
optionally a heat source;
optionally a temperature sensor;
a controller in communication with the pressure transducer, pulse oximeter, optional temperature sensor and optional heat source, wherein the controller is capable of applying a drive signal to the pressure transducer at a frequency below about 0.83 Hz to cause a series of pulsations of a magnitude in the venous blood volume less than diastolic pressure such that the arterial system is minimally disturbed, optionally controlling the temperature of the subject at a second site, and measuring output signals from the pulse oximeter, the output signals containing a component representative of the modulation of venous blood volume due to the series of pulsations; and
a signal processor capable of extracting a value for venous oxygen saturation from the frequency response of the output signals.

13. The apparatus of claim 12, wherein the temperature is above about 30° C.

14. The apparatus of claim 13, wherein the temperature is within a range of from about 30° C. to about 35° C.

15. The apparatus of claim 12, wherein the frequency is within a range of from about 0.1 Hz to about 0.83 Hz.

16. The apparatus of claim 12, wherein the magnitude of the series of pulsations is a pressure within a range of from about 10 mmHg to about 80 mmHg.

17. The apparatus of claim 12, wherein the signal processor is capable of deriving the venous oxygen saturation from the following equations:
$${R}_{Ven} = \frac{{( {\vert APG \vert}_{H z} / {\vert D C \vert}_{0 H z} )}_{RD}}{{( {\vert APG \vert}_{H z} / {\vert D C \vert}_{0 H z} )}_{IR}} ,$$
wherein
R_{Ven} is the modulation ratio related to venous blood,
APG|_{Hz} is the peak Fourier magnitude at the applied pulse frequency of the APG signal,

|DC|_{0 Hz} is the peak Fourier magnitude at 0 Hz frequency of the DC signal; and
SpvO₂=110 −40*R_{Ven}
wherein SpvO₂ is the pulse oximeter estimation of SvO₂.

18. The apparatus of claim 12, wherein the frequency is about 0.2 Hz.

19. The apparatus of claim 12, wherein the magnitude of the series of pulsations is a pressure of about 40 mmHg.
